(19) United States
(12) Patent Application Publication
(10) Pub. No.: US 2023/0024142 A1
(43) Pub. Date: Jan. 26, 2023

(54) APPARATUS FOR CONTROL AND DISTRIBUTION OF NUTRIATED WATER IN AN AUTOMATED AGRICULTURAL FACILITY

(71) Applicant: Iron Ox, Inc., San Carlos, CA (US)

(72) Inventors: Melissa Sobrado Pauly, Mountain View, CA (US); Saqib Naveed, Santa Clara, CA (US); Thomas Kendall, San Jose, CA (US); Sam Walters, San Carlos, CA (US)

(73) Assignee: Iron Ox, Inc., San Carlos, CA (US)

(21) Appl. No.: 17/865,334

(22) Filed: Jul. 14, 2022

Related U.S. Application Data
(60) Provisional application No. 63/225,357, filed on Jul. 23, 2021.

(51) Int. Cl. A01C 21/00; A01G 31/00; A61L 2/24; A61L 2/10

(52) U.S. Cl. CPC A01C 21/007; A01G 31/00; A61L 2/24; A61L 2/10; A01G 2031/006; A61L 2202/11

(57) ABSTRACT

A dosing station comprises a receiving area for receiving a module and a sampling system for acquiring water from the module, analyzing the water and determining a refill recipe based on one or more parameters of the water. The dosing station includes a nutrient dispenser arranged to dispense at least one nutrient into the module according to the refill recipe. The recipe can be based on the type of plants in the module, the age of the plant and/or the physical condition of the plants. The dosing station can change refill recipes for each module within an agricultural facility based on one or more parameters to optimize the nutrient supply for all plants within the agricultural facility.

### CROSS-REFERENCES TO OTHER APPLICATIONS

This application claims priority to U.S. provisional patent application Ser. No. 63/225,357, for “SYSTEM AND METHOD FOR MAINTAINING WATER QUALITY IN MODULES DEPLOYING WITHIN AN AGRICULTURAL FACILITY” filed on Jul. 23, 2021 which is hereby incorporated by reference in entirety for all purposes.

### TECHNICAL FIELD

This invention relates generally to the field of agricultural systems and more specifically to a new and useful system and method for controlling nutriated water in modules deployed within an agricultural facility in the field of agricultural systems.

### BACKGROUND

Global Warming is the long-term warming of the planet's overall temperature. While this warming trend has been going on for a long time, scientists believe almost uniformly that its pace has increased in the last hundred years due to the burning of fossil fuels and other human-induced activities that generate gases which contribute to what scientists refer to as the “greenhouse effect”. Greenhouse gases form a layer in the upper atmosphere of the planet that is more transparent to visible radiation from the sun that to infrared radiation emitted from the planet's surface. Thus, as the sun's radiation warms the surface of the earth, the earth's atmosphere prevents some of the heat from returning directly to space resulting in a warmer planet.

Climate change, which has resulted in changes in weather patterns and growing seasons around the world, is a direct result of global warming. The warming weather can generate extreme weather conditions, such as drought and fires in some areas, more frequent and stronger hurricanes in other areas, tornadoes, flooding and other extreme weather-related events across the globe. The changing weather patterns can also result in adverse changes to our ecosystem. For example, milder winters can allow insects to survive in greater numbers in some areas and/or to emerge earlier in the spring putting additional pressure on trees and plants that can lead to die-off in some instances. As another example, warmer air and ocean temperatures can cause coral bleaching where corals lose their color and die potentially wiping out whole ecosystems that depend on the reefs for food and shelter. Thus, it is imperative that humans reduce, or even reverse, their impact on global warming by reducing their carbon footprint.

Traditional farming techniques have a large impact on our overall carbon footprint. According to some studies, agriculture is the second-leading source of carbon dioxide (CO₂) emissions. For example, traditional farming relies on nitrogen fertilizers produced from manufacturing processes that generate a significant source of greenhouse gases. Crops typically use up only a portion of the nitrogen from fertilizers with the remainder getting broken down by microbes in the soil or finding their way via run off into waterways. Additionally, most vegetable and fruit crops grown by traditional agriculture methods are far away from the ultimate location where they will be consumed. Not only does this require that the crops be trucked long distances, thus burning a lot of fossil fuel in the process, it can also require harvesting crops before they are actually ripe based on an estimated time to delivery lest crops be overly ripe and spoiled upon arrival. These and other factors combine to result in an undesirably high percentage of vegetables and fruit being not suitable for sale and being discarded.

Various efforts have been made to improve upon traditional farming techniques. For example, some companies are growing fruits and vegetables locally, close to or within major population centers, using hydroponics and indoor vertical farming techniques. While these solutions may solve the transportation problem and reduce the amount of fertilizer required, indoor vertical farming is dependent on artificial LED lighting. Energy costs for such farming can be very high, challenging profitability. Also, using fossil-fuel powered electricity undermines the potential environmental benefits of indoor vertical farming. Thus, vertical indoor farming is not an ideal solution to the environmental challenges presented by traditional farming.

Accordingly, new and improved methods and techniques for growing high-quality fruits, vegetables and other food in a sustainable, low-cost manner are desirable.

### BRIEF SUMMARY OF THE INVENTION

Embodiments described herein pertain to a system and method for providing optimized nutriated water to a plurality of grow modules within an agricultural facility. Some embodiments pertain to an integrated system that provides automated testing, mixing and dispensing of nutriated water that is particularly suited to the species, age and/or condition of plants within a grow module. The system can vary the nutriated water in each grow module within the agricultural facility to optimize the growth and productivity of all plants within the facility.

While the embodiments described herein can be useful for providing nutriated water to modules within different types of facilities, some embodiments are particularly useful when included in an automated agricultural facility that includes an autonomous mover for moving grow modules from a growth area to the nutrient system. For example, in some embodiments, plants spend the majority of the time in a growth area where they are exposed to sunlight and consume nutriated water. When the nutriated water becomes depleted, the autonomous mover transports each module to the nutrient dispensing system that tests the water within the module, then dispenses nutriated water according to a recipe suited to the plants within that particular growth module. This process can be repeated numerous times for every growth module within an agricultural facility with little or no human intervention.

In some embodiments a dosing station comprises a receiving area for receiving a module and a sampling system for acquiring water from the module, analyzing the water and determining a refill recipe based on one or more parameters of the water. A nutrient dispenser is arranged to dispense at least one nutrient into the module according to the refill recipe. In various embodiments the sampling system includes an electrical resistivity probe.

In some embodiments the sampling system includes an intake pipe that is arranged to be immersed in the water in the module. In various embodiments the dosing station further comprises a sanitization system arranged to sanitize the intake pipe after the immersing. In some embodiments the sanitization system comprises an ultraviolet source. In various embodiments the intake pipe is tapered and the ultraviolet source is axially aligned with the intake pipe. In some embodiments the module is a first module and the receiving area is arranged to receive a second module. In various embodiments the refill recipe is determined at least in part from an optical image of one or more plants in the module.

In some embodiments a method of refilling a grow module in an agricultural facility comprises positioning the grow module adjacent a dosing station, acquiring a water sample from the grow module with a sampling system, testing the water sample and generating an output based on the water sample. The method may further comprise determining a grow module refill recipe based on the output and dispensing a nutrient into the grow module based on the refill recipe. In various embodiments the sampling system includes an electrical resistivity probe. In some embodiments the sampling system further comprises an intake pipe that extends into the grow module to acquire the water sample.

In some embodiments the method further comprises sanitizing the intake pipe after acquiring the water sample. In various embodiments the refill recipe is determined at least in part based on a type of plant in the grow module. In some embodiments the refill recipe is determined at least in part from an optical image of one or more plants in the grow module.

In some embodiments a method of growing plants in a grow module within an agricultural facility comprises positioning the grow module in a growth area of the agricultural facility, transporting the grow module to a dosing station, acquiring a water sample from the grow module with a sampling system, testing the water sample and generating an output based on the water sample. The method may further include determining a module refill recipe based on the output, dispensing a nutrient into the grow module based on the refill recipe and transporting the grow module to the growth area.

In some embodiments the transporting is performed by an autonomous mover. In various embodiments the refill recipe is additionally based on a species of the plants in the grow module. In some embodiments the refill recipe is additionally based on an image of the plants in the grow module. In various embodiments the output indicates a nutrient content in the water sample. In some embodiments the output indicates a pH of the water sample.

### BRIEF DESCRIPTION OF THE DRAWINGS

FIG. 1 is a schematic representation of the layout of an example agricultural facility in which some embodiments disclosed herein can be used;

FIGS. 2A and 2B are simplified isometric front and rear views, respectively, of a dosing station for testing and refilling modules with nutriated water, that can be used in the agricultural facility disclosed in FIG. 1;

FIG. 3 illustrates a simplified side view of the dosing station shown in FIGS. 2A and 2B during an initial stage of a module refill operation;

FIG. 4 illustrates a simplified side view of the dosing station shown in FIGS. 2A-3 with a robotic manipulator that has elevated the raft out of the module;

FIG. 5 illustrates a simplified side view of the dosing station shown in FIGS. 2A-4 with the raft at an elevated position and the dosing station extended over the module;

FIG. 6 illustrates a simplified side view of the dosing station shown in FIGS. 2A-5 with the dosing station in a retracted position and the raft replaced in the module;

FIG. 7 illustrates steps associated with a method 700 of operating the dosing station described in FIGS. 2A-6 to test and refill the module;

FIG. 8 illustrates a simplified plan view of the module and rafts described in FIGS. 1-7;

FIG. 9 illustrates a simplified isometric view of one example of a water sampling system that can be used in the dosing station described in FIGS. 1 and 2A-7;

FIG. 10 illustrates a simplified cross-sectional view of the reservoir of the water sampling system shown in FIG. 9;

FIG. 11 illustrates a simplified view of a sample pipe cleaning system that can be used in the dosing station described in FIGS. 1 and 2A-7;

FIG. 12 illustrates a simplified cross-sectional view of the sample pipe shown in FIG. 11 during a cleaning operation;

FIG. 13 illustrates a simplified view of an example nutrient dispensing system that can be used in the dosing station described in FIGS. 1 and 2A-7; and

FIG. 14 illustrates a simplified partial cross-sectional view of an example spout assembly described in FIG. 13.

### DETAILED DESCRIPTION OF THE INVENTION

The following description of embodiments of the invention is not intended to limit the invention to these embodiments but rather to enable a person skilled in the art to make and use this invention. Variations, configurations, implementations, example implementations, and examples described herein are optional and are not exclusive to the variations, configurations, implementations, example implementations, and examples they describe. The inventions described herein can include any and all permutations of these variations, configurations, implementations, example implementations, and examples.

Embodiments described herein pertain to a system and method for controlling nutriated water in agricultural modules to optimize the growth of plants within an agricultural facility. Some embodiments pertain to an integrated automated dosing system that samples the depleted water in agricultural modules, determines adjustments that need to be made in water level and nutrient concentrations to optimize the health and growth of the particular plants in that module, then dispenses nutriated water with appropriate concentrations such that when mixed with the depleted water within the module provides the optimum concentration of nutrients for the plants within that module. The movement of the modules to the dosing station, the sampling of the water, the determination of the optimum nutrient levels, the dispensing of the optimum concentration of nutrients and the return of the module to a growth area can be completely automated with little to no human intervention.

### Example Agricultural Facility

While the embodiments described herein can be useful for using a dosing station to refill grow modules with nutriated water in many different types of facilities or farms, some embodiments are particularly useful when included in an agricultural facility that is highly automated.

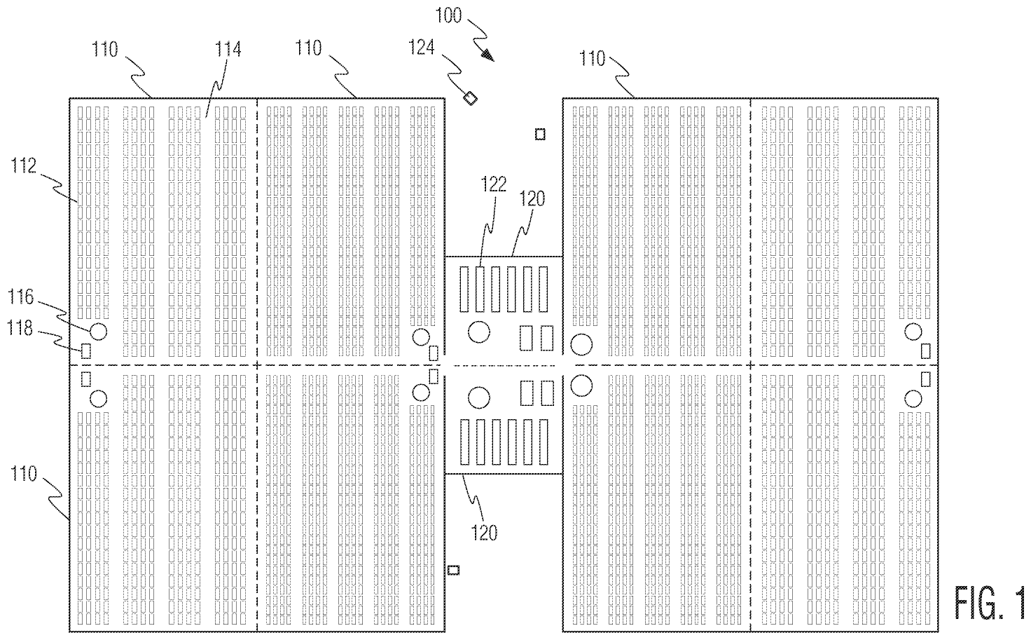

FIG. 1 is a schematic representation of the layout of an example of an agricultural facility 100 in which some embodiments disclosed herein can be used. It is to be understood that agricultural facility 100 is set forth herein for illustrative purposes only and that embodiments of the inventions disclosed herein can be used in agricultural facility 100 as well as many other types of agricultural facilities.

As shown in FIG. 1, agricultural facility 100, which can include one or more greenhouses in which different portions of the agricultural facility reside, includes one or more farming blocks 110 along with one or more processing bays 120. The farming blocks 110 and processing bays 120 can be connected to each other, for example by hallways and/or common doors, so that plants, equipment, materials, personnel and other items can move between the farming blocks and processing bays efficiently and without exiting the agricultural facility. To enable automation in the growth and harvesting of plants within facility 100, in some embodiments, movers (e.g., autonomous vehicles 124) can be provided that can be programmed or otherwise configured to autonomously move plants within agricultural facility 100 including moving plants within the various farming blocks 110, within the processing bays 120 and/or between the farming blocks 110 and the processing bays 120 without exiting the facility. Each mover 124 can be an autonomous robot, such as described in U.S. Pat. No. 10,813,295 entitled “System and Method for Automating Transfer of Plants within an Agricultural Facility”, which is incorporated by reference herein in its entirety.

Generally, each processing bay 120 includes multiple stations (represented by various geometric shapes in FIG. 1 within the bays 120) in which various phases of the agricultural cycle occur. For example, within a given processing bay 120 can be separate stations that seed, germinate, pot, harvest, and/or package plants. In some embodiments, each processing bay 120 can function as an autonomous assembly line in which the multiple stations are automated and connected by conveyors and/or an autonomous mover 124. The multiple automated stations can cooperate to seed, germinate, pot, harvest, and package plants with minimal human intervention or without any need for a human laborer to handle the plants during those processes. Hence processing bays 120 are also referred to herein as “automation bays 120”.

Plants can be automatically transported to the farming block 110 (e.g., by a mover 124) after being initially propagated from a seed to a seedling in one of the automation bays 120 (e.g., in a propagation system 122). The farming blocks 110 can then function as longer-term holding bays for plants between the potting and harvest stages. Each farming block 110 can be configured as an enclosed, environmentally-controlled grow area in which plants, after being transferred from the processing bay at an intermediate stage of growth, are provided with nutrients and light that they require to mature until the harvesting stage. Thus, a given plant can spend the majority of the agricultural cycle (e.g., the time between the potting and harvesting stages for the plant) in one of the farming blocks 110. In some embodiments, the farming blocks 110 can include controls and equipment to autonomously control growth input parameters (e.g., light, temperature, nutrients, etc.) to grow the plants under conditions that have been determined to be ideal for the particular plants.

The plants can be grown within farming blocks 110 in modules 112 that are distributed across the farming block and separated by aisles 114 accessible by the autonomous mover 124. In some embodiments, the modules 112 are distributed throughout the farming block 110 in a horizontal arrangement so that the plants in each module have full access to natural light that enters the farming block through greenhouse windows around the sides and roof of the farming block. In some implementations, farming blocks 110 can grow plants using hydroponic techniques and each module 112 can include multiple rafts that float or partially float on a bed of nutrient-rich water within the module.

Each farming block 110 can also include, among other stations and/or equipment, a module imaging station 116 and a dosing station 118. The module imaging station 116 can be configured to scan whole modules or individual rafts or trays within a module in the farming block, while the dosing station 118 can be configured to add water and nutrients to modules in the farming block. For example, a mover 124 dedicated to the farming block can intermittently: deliver modules in the farming block to the module imaging station, 116 which scans plants in the module and characterizes qualities of these plants and pest pressures in the module based on features detected in these images; and deliver modules in the farming block to the dosing station 118, which tests the water in the module and refills the module with water having a nutrient recipe optimized for the particular plants in that module. In some embodiments data from the imaging station 116 can be used to modify the nutrient recipe for that particular module to optimize growth on a module-by-module basis. The optimization of nutrients on a module-by-module basis results in increased yields and growth rate as compared to vertical hydroponics techniques that typically use a common reservoir of nutriated water for all plants within a relatively large growth area.

While agricultural facility 100 is depicted in FIG. 1 as including eight separate farming blocks 110 and two automation bays 120, it is to be understood that this is for illustrative purposes only. In other embodiments facility 100 can include fewer or more than eight farming blocks 110 and can include fewer or more than two automation bays 120. Additionally, while agricultural facility is depicted with a farming block to automation bay ratio of 4:1, this ratio is also to be understood as serving illustrative purposes only and other embodiments can include ratios lower than or higher than 4:1.

Agricultural facility 100 can be designed in a manner where the approach to growing fruits, vegetables and other plants has been completely rethought as compared to traditional farming techniques. For example, farming blocks 110 can represent the majority of the physical space of agricultural facility 100. Plants can spend the majority of their growth cycle in the farming blocks where they are grown with hydroponics techniques while being arranged throughout the farming blocks horizontally. Hydroponic growth methods can produce higher quality crops with better yields using less labor than traditional soil-based farming while also providing a large savings in terms of water and fertilizer as compared to traditional, soil-based farming techniques. The horizontal distribution of plants within the farming blocks 110 differs from the vertical arrangement of plants that is used in some indoor hydroponics farming facilities that require large banks of artificial lights to adequately provide artificial sunlight for the plants. Such a horizontal arrangement can thus provide a large energy saving as compared to indoor vertical hydroponics farming techniques as well as lending itself to increased automation.

Further, instead of having a set of greenhouses defining primary operating areas within a facility, the combination of farming blocks 110 and processing bays 120 allow the processing bays 120 to function as the primary operating area of agricultural facility 100 with farming blocks 110 acting as automated greenhouses and functioning as infeed, outfeed, and holding zones for product flowing through the automation bays. For example, the agricultural facility can include one processing bay 120 and a set of farming blocks 110 arranged radially about the processing bay, each connected to the processing bay by a path traversable by the autonomous mover 124 to transfer plants between the processing bay and the farming block.

Various sensors can be positioned throughout agricultural facility 100 to monitor all phases of and aspects of plant growth within both farming blocks 110 and processing bays 120. Non-limiting examples of such sensors can include: water quality sensors (e.g., sensors that monitor water temperature, water level, dissolved oxygen, pH, and/or nutrient levels), air quality sensors (e.g., sensors that monitor ambient air temperature, air speed, light level, relative humidity, carbon dioxide levels, oxygen levels, and the like) and pest sensors, among others. Data from the sensors can be collected, analyzed and used, in conjunction with the automated features of the agricultural facility, to ensure that each individual plant receives the optimal levels of sunshine, water, and nutrients. This level of precision towards the inputs used in the growth cycle, which is not readily obtainable with traditional agricultural techniques, can lead to the consumption of less water and energy and to the reduced emission of CO₂ than traditional farming techniques while also generating less waste at every stage of the growth process. Additionally, the data that can be continually collected from the plants grown within the facility can be used to constantly refine the growth and harvesting processes and improve both plant quality and quantity thereby reducing environmental impacts. For example, tracking precise levels of nutrients, water levels, temperature, and humidity, can lead to the creation of data models that can enable improved crop yield estimates and/or techniques to increase growth rates. The improved estimates can enable more precise yields so that agricultural facility 100 can respond to customer orders in a more accurate manner leading to less waste since what is shipped is what was ordered. The collected data can also be used to adjust plant growing conditions in a manner that results in improvements in the growth rate and/or yield of each plant. Such efficiency gains can then allow greenhouses to produce more produce in a given space. The increased growth rate results in a faster time to harvest, which increases overall efficiency of the facility.

Within the farming blocks 110 of agricultural facility 100, plants can be spaced apart from each other at a first spacing density that allows room for the plants to grow to their desired size prior to harvesting. However, during the early stages of their growth cycle the plants are located in one of the processing bays 120. Since the plants are much smaller at their early growth stage, prior to be transferred to a framing block, in some implementations the plants can be propagated within a propagation system 122 that works with plants at a second spacing density where the plants are much more closely spaced together prior to being transplanted into the modules and transported to the farming blocks. That is, the second spacing density can be much denser than the first spacing density. For example, in some embodiments, plants can be propagated from a seed to seedling stage within plugs that are physically touching each other in the propagation system 122 or spaced less than a couple of millimeters (or less than 1 mm) apart from each other. After the plants reach the seedling stage, they can then be transferred to a potting system (e.g., a raft or a tray) where they are spaced apart several inches or more depending on the type of plant (e.g., basil plants can be transplanted into a potting system that might have a first spacing (in terms of inches) between adjacent basil plants while certain types of lettuces might grow better in a potting system that has a second spacing between plants that is greater than the first spacing.

Certain embodiments disclosed herein pertain to new and improved dosing stations that that maintain nutriated water within grow modules, such as dosing station 118, that can monitor and control uniquely tailored nutrient levels for each individual tray of a grow module as disclosed herein and discussed in more detail below.

### Dosing Station

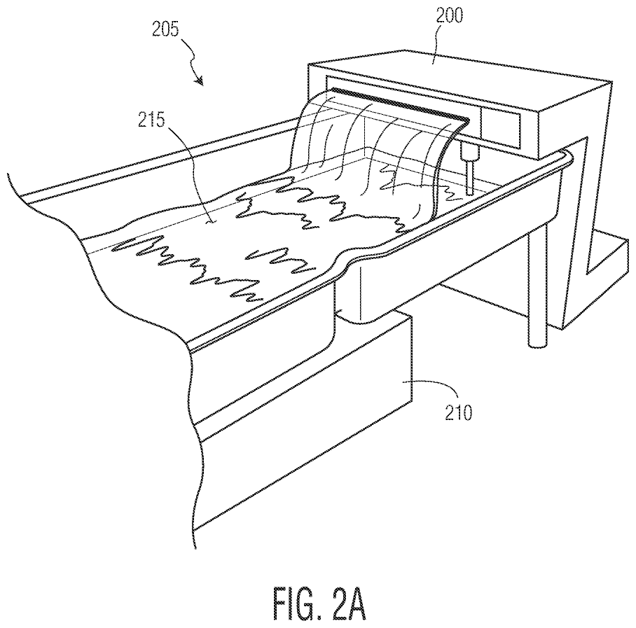

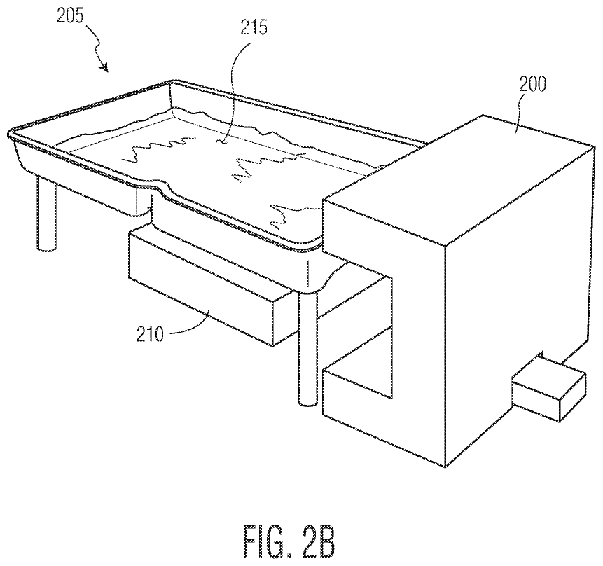

FIGS. 2A and 2B are simplified isometric front and rear views, respectively, of a dosing station for testing and refilling modules with nutriated water, according to some embodiments. Dosing station 200 is representative of dosing station 118 discussed with respect to FIG. 1 and, in some implementations, can reside within an agricultural facility, such as within one of the farming blocks 110 of agricultural facility 100. As shown in FIGS. 2A and 2B, dosing station 200 can service a plurality of grow modules 205 that are brought to the dosing station via autonomous mover 210.

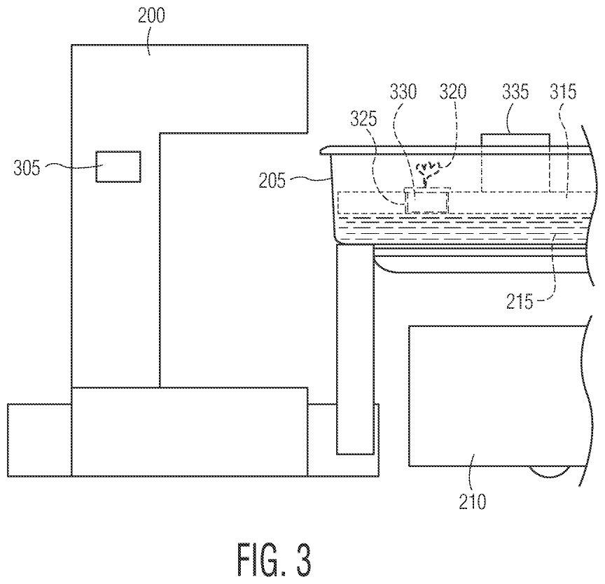

Dosing station 200, of which various views are also shown in FIGS. 3-14, can be equipped with components and features that enable the testing of water 215 within a grow module 205 and the refilling of the water with a particular nutrient recipe optimized for the particular plants in that module with little to no human intervention in the process.

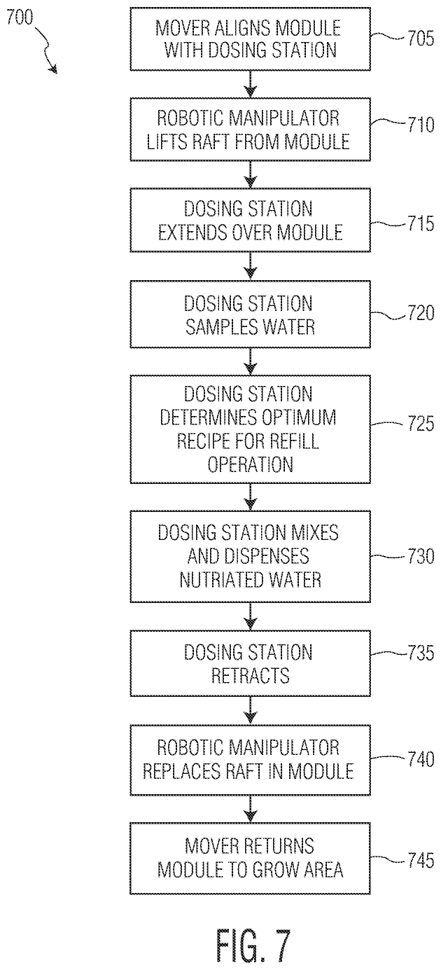

FIGS. 3-6 illustrate simplified sequential side views of a dosing station interfacing with a module to test and refill the module with nutriated water, according to embodiments of the disclosure. FIG. 7 illustrates steps associated with a method 700 of operating the dosing station to test and refill the module as shown in FIGS. 3-5. FIGS. 3-7 will now be described simultaneously.

FIG. 3 illustrates a simplified side view of dosing station 200 during an initial stage of a refill operation. As shown in FIG. 3 (step 705), mover 210 supports module 205 and approaches dosing station 200. In some embodiments dosing station 200 includes a module location sensor 305 that can be used to provide a command to autonomous vehicle 210 to stop at an appropriate distance from dosing station 200 (e.g., within a receiving area of dosing station), however in other embodiments autonomous vehicle 210 includes one or more distance sensors that command the autonomous vehicle to stop at an appropriate location. In some embodiments module location sensor 305 is an optical, ultrasonic, hall-effect or other suitable type of sensor. As further shown in FIG. 3, nutriated water 215 is relatively low in module 205 and has been brought to dosing station 200 for refilling. A raft 315 floats on nutriated water 215 such that plant 320, which is retained in perforation 325 by cup 330 is at least partially immersed in the nutriated water. In some embodiments a tube 335 can be positioned on raft 315 to keep foliage of plants 320 away from effector locations on the raft so a robotic manipulator (not shown in FIG. 3) can access raft 315, as described in more detail below.

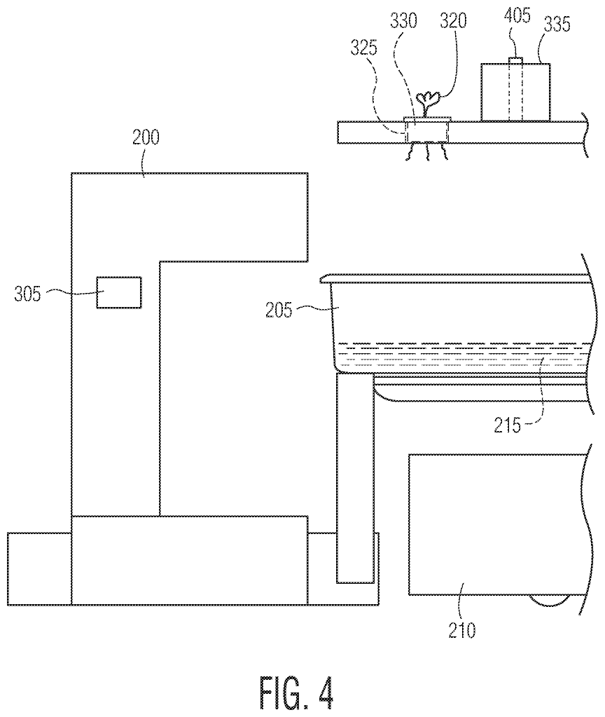

As shown in FIG. 4 (step 710) a robotic manipulator (not shown in FIG. 4) has aligned an end effector 405 within tube 335. In some embodiments end effector 405 is a vacuum or mechanical mechanism that attaches to raft 315. Robotic manipulator can be any type of robot or mechanism that can align to raft 315 via a fiducial (not shown in FIG. 4) or other means and lift raft to a height that allows access to module 205 by dosing station 200.

A robotic raft manipulator can also be arranged near the dosing station, and the mover can arrange a module in a module dock adjacent the robotic raft manipulator in order to enable the robotic raft manipulator to navigate its end effector 405 to a raft 315 in the module 205 and to lift or remove the raft from the module, thereby enabling the dosing station to extend a sample pipe into the module and to disperse water via a spout (or spigot, waterfall)—directly into the module without upsetting the raft or plants contained in the module. In some embodiments, a module 205 can include multiple (e.g., two, four) rafts 315 that cover a large proportion (e.g., more than 97%) of the surface area of water in the module. In various embodiments, the robotic raft manipulator can: detect an optical fiducial on a raft; navigate its end effector (e.g., a set of suction wands) to a flat region on the raft defined relative to the optical fiducials; lift the raft (and plants contained therein) to expose water within the module to the dosing station.

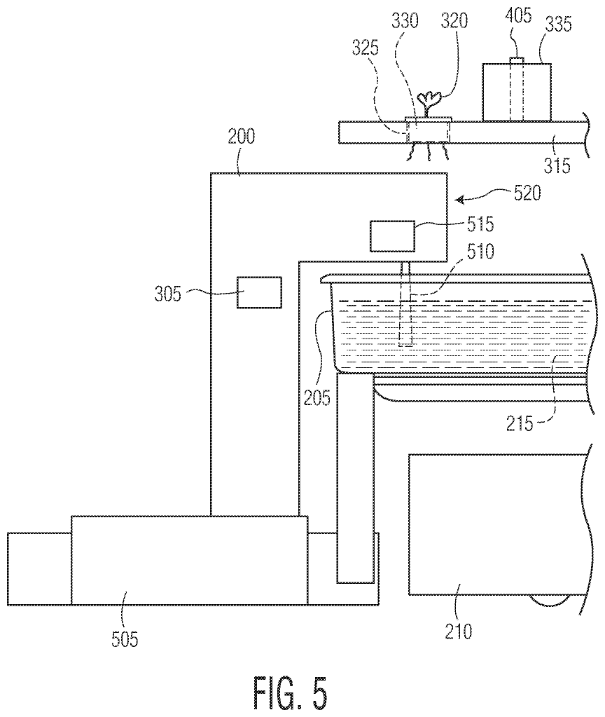

As shown in FIG. 5 (step 715) while robotic manipulator (not shown in FIG. 5) retains raft 315 at an elevated position, dosing station 200 extends via a slide mechanism 505 over module 205. In some embodiments a sensor, such as sensor 305 can control the distance dosing station 200 extends. As further shown in FIG. 5 (step 720) dosing station 200 extends a sample pipe 510 into module 205. In some embodiments sample pipe 510 may rotate or otherwise be extended to access water 215 in the module.

Dosing station 200 can also detect a level of water 215 in module 205 using a fill level sensor 515 that can be an ultrasonic, optical, electrical or other type of sensor. In one embodiment, fill level sensor 515 includes an infrared or ultrasonic sensor directed downwardly toward the module 205 and is configured to output a signal representing a distance from a surface of water in a module present in module 205. In some embodiments dosing station 200 can store a standard height of modules 205 deployed in the facility and can calculate a fill level of water in the module (e.g., in inches, millimeters) based on an output of the fill level sensor 515 and the standard module height. Therefore, dosing station 200 can detect the fill level of water in the module 205 without contacting the module or water contained therein to minimize cross-contamination of modules.

As further shown in FIG. 5 (step 720), dosing station 200 intakes water 215 via sample pipe 510 from module to determine the nutrient levels, pH, oxygenation, bacteria count or any other suitable metrics of the depleted water. Dosing station 200 may have a sample reservoir (not shown in FIG. 5) that includes a plurality of sensors to detect parameters of water 215 in its depleted condition.

In step 725, dosing station 200 or other computing device (not shown in FIG. 5) can use the parameters of water 215, among other inputs, to determine an optimum refill recipe for the particular plants 320 in the particular module 205. More specifically, in some embodiments inputs from an imaging station, such as imaging station 116 illustrated in FIG. 1, that uses images of the foliage and/or root systems of the particular plants 320 in the particular module 205 (or tray, not shown in FIG. 5) along with the parameters of water 215 in its depleted state can be used to determine an optimum refill recipe for the particular plants 320.

In step 730, dosing station 200 mixes and dispenses nutriated water in accordance with the optimum recipe determined in step 725. More specifically, in some embodiments dosing station calculates that amount of water that needs to be added to module 205 to refill it and adjusts the nutrient levels in the dispensed nutriated water such that when the dispensed via spout 520, the nutriated water mixes with the depleted water in module 205 resulting in an optimum level of nutrients in the mixed water within the module. Thus, dosing station 200 can be used to optimize the nutrient levels for plants 320 in each particular module according to their specific needs resulting in highly controlled and optimized growing conditions for myriad different plants within a sizable agricultural facility 100 (see FIG. 1).

For example, if plants 320 are nitrogen deficient showing a pale yellow green color, that is for example, detected by an imaging station (e.g., imaging station 116 in FIG. 1) dosing station 200 can be used to refill module 205 with nutriated water that is relatively high in nitrogen. Similarly, if plants 320 are phosphorous deficient showing dull blue green color and stunted root growth that is, for example detected by an imaging station (e.g., imaging station 116 in FIG. 1) dosing station 200 can be used to refill module 205 with nutriated water that is relatively high in phosphorous. Thus, dosing station 200 provides the ability to finely tune the nutrients available to individual groups of plants to optimize growth conditions on a module-by-module 205 basis. Used in combination with an autonomous mover 210 and, optionally, an imaging station 116 (see FIG. 1), the process can be fully automated resulting in significant growth rate and yield increase for an entire agricultural facility 100 (see FIG. 1). When used in conjunction with artificial intelligence, the nutrient recipe can be continuously refined to optimize plant health and growth rate for myriad plant species.

In an optional step, after dispensing the nutriated water, dosing station 200 can intake a new sample of water from module 205 and test it to determine if it has the expected final nutrient levels. In some embodiments the nutrient mixing may be performed in an ancillary system that is coupled to dosing station for dispensing the nutriated water while in other embodiments the nutrients my be injected into a water supply line that delivers water directly to module 205.

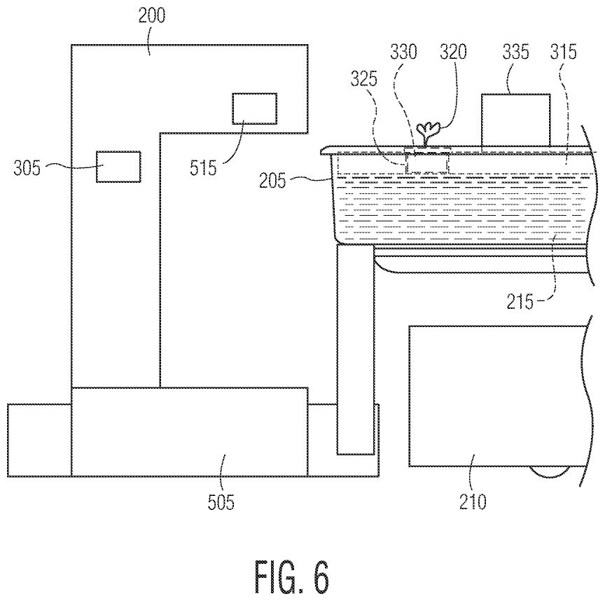

As shown in FIG. 6 (step 735) dosing station 200 retracts sample pipe 510, then retracts horizontally via slide mechanism 505 so it is out of the way for raft 315 to be replaced within module 205 (step 740) by the robotic manipulatory. In some embodiments when retracted sample pipe 510 may be cleaned using a UV treatment or chemical treatment to mitigate spreading bacteria or other contaminants between modules 205. In various embodiments the robotic manipulator (not shown) may be located in a separate location away from dosing station 200 such that automated mover 210 moves module 205 under the robotic manipulator which raises raft 315, then the automated mover moves only the module to dosing station 200. After raft 315 is replaced in module 205, automated mover 210 returns the module to the grow area (step 745). In some embodiments dosing station 200 may be configured to recycle and clean water 315 in module 205 by intaking a significant quantity of the water, filtering it, reconditioning it, testing it, then dispensing cleaned and reconditioned water to the module 205, along with any additional water, nutrients or other suitable additives.

It will be appreciated that method 700 is illustrative and that variations and modifications are possible. Steps described as sequential may be executed in parallel, order of steps may be varied, and steps may be modified, combined, added or omitted.

In some embodiments dosing station 200 is located within an agricultural facility and includes: a module dock configured to receive a module delivered autonomously by a mover; a robotic raft manipulator configured to temporarily remove a raft loaded with plants from the module; a sample pipe configured to insert into a volume of water contained in the module; a sample reservoir; a sample pump configured to draw water from the module into the sample reservoir via the sample pipe; a suite of sensors coupled to the sample reservoir and configured to output signals representative of characteristics of water in the sample reservoir and therefore in the module; a spout coupled to a freshwater supply and configured to dispense water into the module; and a suite of nutrient containers and pumps (or valves) configured to meter nutrients into the module, such as via the spout.

In some embodiments the dosing station 200 can: actuate the sample pipe and the sample pump to acquire a water sample (e.g., a one-gallon sample) from a module; test characteristics of the water sample; characterize differences between actual and target characteristics of water in the module; and then automatically dispense additional water and/or nutrients into the module to reduce this difference. For example, the dosing station can retrieve a nutrient recipe assigned to the module based on types, stages, and/or ages of plants contained in the module.

In various embodiments, the dosing station (or other system(s) within the agricultural facility) can: capture photographic images of plants and/or roots in the module; estimate nutrient overages or insufficiencies based on visual characteristics of these plants; tune or generate a custom nutrient recipe for these plants; and then automatically dispense additional water and/or nutrients into the module in order to achieve this custom nutrient recipe in the module. Therefore, the dosing station can cooperate with the mover to maintain nutrient-rich water conditions within numerous modules within the agricultural facility according to recipes assigned to or customized for the particular plants occupying the modules.

In some embodiments dosing station 200 can execute this process with only a single point of physical contact between the dosing station and water contained in the module (i.e., the sample pipe), thereby limiting opportunity for pathogen spread between modules served by the dosing station via wet surfaces on the dosing station, as described in more detail below.

In various embodiments the dosing station can also maintain a water sample in the sample reservoir between refill cycles such that water quality sensors in the dosing station remain fully bathed in water between refill cycles, thereby extending calibration and viability of these sensors over time and eliminating a need to sanitize these sensors between refill cycles, as described in more detail below.

As shown in FIGS. 1-6, mover 210 (e.g., a wheeled autonomous vehicle or “loader”) is configured to navigate autonomously throughout the agricultural facility: to relocate modules to a module docking location adjacent a transfer station in preparation for loading plants into or unloading plants from these modules; to selectively return these modules to their assigned grow locations throughout the agricultural facility; and to selectively cycle these modules through the dosing station verification and correction of fill, nutrient, and pH levels in these modules. In particular, the mover can be configured to automatically navigate throughout the agricultural facility to a particular location under or near a module, to couple to or lift the module, to navigate with the module to the dosing station within the agricultural facility, and to release (or “deposit”) the module at the dosing station.

### Modules, Trays and Rafts

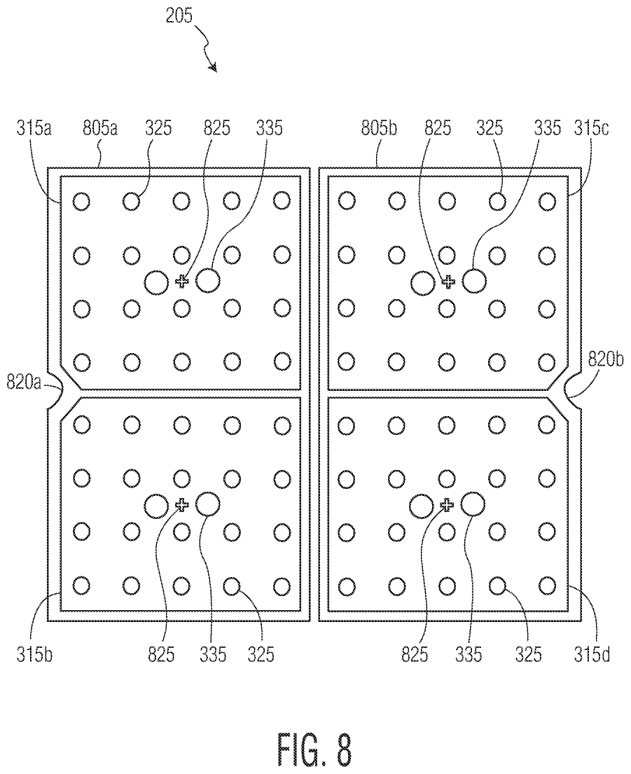

FIG. 8 illustrates a simplified plan view of the example module 205 described in FIGS. 1-7, according to some embodiments. Module 205 is representative of module 112, discussed with respect to FIG. 1, and, in some implementations, can reside within an agricultural facility, such as within one of the farming blocks 110 of agricultural facility 100. As shown in FIG. 8, module 205 can include a first tray 805a and a second tray 805b. In this particular embodiment, each tray 805a, 805b can accommodate two rafts where first and second rafts 315a, 315b, respectively, are within first tray 805a and third and fourth rafts 315c, 315d, respectively are within second tray 805b. In the embodiment shown, each raft 315a-315d can accommodate 20 individual plants with one plant in each perforation 325. Although module 205 illustrates two trays with two rafts each where each tray has 20 perforations, other embodiments may include other suitable variations of the number of rafts, trays and perforations.

In this particular embodiment each tray 805a, 805b is self-contained and holds a common reservoir of nutriated water 215 (see FIG. 6) for the plants that are supported by the respective rafts in each tray. Therefore, dosing station 200 (not shown in FIG. 8) can be used to control a particular nutrient recipe for a set of 2 rafts holding 40 plants, in this particular embodiment. For example, if first and second rafts 315a, 315b have plants that are nitrogen deficient showing a pale yellow green color, that is for example, detected by an imaging station (e.g., imaging station 116 in FIG. 1) dosing station 200 can be used to refill tray 805a with nutriated water that is relatively high in nitrogen. Similarly, if second and third rafts 315c, 315d have plants that are phosphorous deficient showing dull blue green color that is, for example detected by an imaging station (e.g., imaging station 116 in FIG. 1) dosing station 200 can be used to refill tray 805b with nutriated water that is relatively high in phosphorous. Thus, dosing station 200 provides the ability to finely tune the nutrients available to individual groups (e.g., 10, 20, 40, 80) of plants to optimize growth conditions on a tray-by-tray 805a, 805b basis. Used in combination with an autonomous mover 210 and, optionally, an imaging station 116 (see FIG. 1), the process can be fully automated resulting in significant growth rate and yield increase for an entire agricultural facility 100 (see FIG. 1).

As further shown in FIG. 8 in some embodiments trays 805a, 805b can each have one or more orientation features 820a, 820b that hold rafts 315a-315d in place. Each module 205 can have one or more legs or supports (not shown in FIG. 8) to provide space underneath for autonomous mover 210 to move underneath the tray and lift the tray. Each tray 805a, 805b can also include one or more splash guards and other features to minimize the water from splashing out of the tray during movement by autonomous mover 210. In some embodiments each raft 315a-315d can be blow molded and formed from polypropylene, polyethylene or other suitable material for ease of cleaning and may have a sealed interior space containing air for buoyancy.

In some embodiments each raft 315a-315d can include one or more fiducials 825 (e.g., optical alignment marks) that enable a robotic manipulator to attach one or more end effectors in the regions guarded by tubes 335.

In various embodiments, an agricultural facility, such as facility 100 in FIG. 1, includes a set of modules 205 configured to house a group of plants throughout a segment of the growth cycle of the plants (e.g., four weeks of a twelve-week grow-period) and configured for mobile deployment between grow locations, a transfer station, a cleaning station, and/or the dosing station 200 by the autonomous mover 210 over time. Each module 205 can define a standard size (e.g., four feet in width by eight feet in length by four feet in height; two meters in width by five meters in length by one meter in height) and can include any suitable number of perforations 315 (e.g., plant slots) matched to the segment of plant growth cycle associated with the particular module. For example: a seeding-type module can include 192 plant slots; a nursing-type module can include 48 plant slots (i.e., one-quarter as many as seeding-type modules); and a finishing-type module can include twelve plant slots (i.e., one-quarter as many as nursing-type modules); despite these modules 205 having the same overall size and geometry.

In one embodiment, a module 205 includes: a set of hydroponic trays (or hydroponic tubes), each defining a (linear) array of plant slots, wherein each plant slot is configured to receive and retain one plant (or one cluster of multiple plants); a carriage or frame supporting the set of hydroponic trays at an angle, such as declining 5° from horizontal; a reservoir fluidly coupled to the set of hydroponic trays and configured to collect water flowing out of the hydroponic trays; and a pump configured to cycle water from the reservoir back through the set of hydroponic trays. The module can additionally or alternatively be configured to transiently connect to a water supply line and to a water return line in the agricultural facility, which can provide a constant supply of water and nutrients to plants in this module. In this embodiment, the module can also include: an optical fiducial at the front of each hydroponic tray; optical fiducials at each end of each hydroponic tray; an optical fiducial adjacent each plant slot along each hydroponic tray; and/or optical fiducials at three or four corners of the modules; etc. The mover, a robotic plant manipulator at a transfer station, and/or a robotic raft manipulator at the dosing station, etc. can thus detect these optical fiducials, such as through optical sensors, integrated into these subsystems to identify and locate the module and to locate plant slots in each hydroponic tray in the module.

In another embodiment, a module includes: an open tray configured to contain a standing volume of water and nutrients; a cover arranged over the open tray and including a set of perforations, wherein each perforation defines a plant slot configured to receive and retain one plant (or one cluster of plants); and a stand configured to support the tray off of the ground. In the implementation: the open tray can define a standard rectangular geometry, as described above; and the raft can include a rectangular cover configured to float in water in the tray. For example, the raft can include: a rigid panel (e.g., nylon or aluminum sheet) defining an array (e.g., a linear grid array, a close-pack array) of plant slots; and floats extending across the underside of the rigid panel and exhibiting sufficient buoyancy and/or height to maintain an air gap between the top surface of water in the tray and the bottom surface of the raft when the array of plant slots in the raft are filled with plants, thereby maintaining exposure to air, and therefore oxygen, for upper root systems of these plants. Furthermore, in this example, because the raft floats on the water in the tray, the raft can ensure that roots of these plants remain in contact with water in the tray despite changes to the water level in the tray. In another example, a raft includes a hollow polymer structure (e.g., a blow molded or roto-molded plastic structure) configured to float directly on a volume of water within the tray and defining a set of perforations that form an array of plant slots configured to support and retain a set of plant cups.

In various embodiments, a tray can also define a set of key features configured to constrain a raft in a particular orientation within the tray. For example, the tray can define a rectangular interior volume (e.g., a 3.1 feet-wide, 6.2 feet-long, 8 inch-deep section) with a pair of opposing key features located along the two long sides of the interior volume and extending inwardly toward the center of the interior volume. In this example, each raft can include a structure half the length of the interior volume of the tray and defining two chamfered corners configured to engage the opposing key features in the tray (e.g., a 3 foot-wide by 3 foot-long raft with two 3 inch-chamfered corners). Thus, in this example, the tray can be loaded with two rafts constrained and separated by the key features of the tray.

For example, a robotic plant manipulator can load the tray with: a first raft defining a first array of plant slots at a first density and containing a first set of plants at a first growth stage; and a second raft defining a second array of plant slots at the first density and containing a second set of plants at the same growth stage. Therefore, in this example, the robotic plant manipulator can: receive multiple smaller, empty rafts, such as via a conveyor, and fill plant slots in these rafts with plants before these rafts are loaded into a single tray; and later receive these populated rafts, such as via the same conveyor, and remove plant from these rafts for harvest or transfer to other rafts with lower-density arrays of plant slots. Thus, the robotic plant manipulator can interface with smaller rafts, thereby reducing a minimum reach of the robotic plant manipulator and reducing a footprint of the transfer station with minimal or no change in maximum plant density at each module. Furthermore, keying features in a tray can maintain the location of a second raft in the tray while a first raft is removed by a robotic raft manipulator (described below), thereby enabling the robotic raft manipulator to return to, quickly engage, and similarly remove the second raft from the tray, such as without directly sensing or detecting the second raft.

In some embodiments, the module can include a set of optical fiducials arranged on the top surface of the raft and/or the tray and configured to indicate position, orientation, distance, type, and/or unique identity of the module. For example, the module can include: one optical fiducial (e.g., a unique barcode or quick-response code) arranged at each of three or four corners on the raft; three (identical) colored dots (e.g., yellow for nursery stage, red for finishing stage) arranged at corners of the raft or tray; or one optical fiducial adjacent each plant slot on the raft (e.g., a colored circle, square, or polygon of known geometry and dimension encircling each plant slot); etc.

In various embodiments the module can include an open tray with a fixed raft (or “lid”). In this implementation, the tray and fixed raft can define geometries and features similar to those in the foregoing embodiment but with the raft fixedly coupled to the rim of the tray, such as sealed against the rim of the tray to prevent water from splashing out of the tray when the module is moved by the mover.

In some embodiments that employ an overhead autonomous mover, the frame supporting the hydroponic tubes or tray can include a set of hard points that the mover is configured to engage when moving the module between its assigned grow location on the floor of the agricultural facility and a module docking location adjacent a transfer station or dosing station. The mover can autonomously navigate over the module, detect the module from overhead, and lift the module before moving the module laterally; in this implementation, the module can include optical fiducials arranged across the top side of the tray, raft, or hydroponic tubes, etc. such that these optical fiducials may be detected by the mover and thus enable the mover to align itself over the module before lifting the module from hard points on the frame.

In another embodiment the autonomous mover can be configured to autonomously navigate under the module. In this implementation, the module can include optical fiducials arranged across the underside of the frame, tray, or hydroponic tubes, etc. such that these optical fiducials may be detected by the mover and thus enable the mover to align itself under the module before engaging the module.

In another embodiment, the module can include: a set of wheels, casters, or rollers, etc.; a latch on a side or rear of the frame; and optical fiducials adjacent the latch. The mover can thus detect these optical fiducials to align itself to the latch, engage the latch accordingly, and then pull or push the module between its assigned location on the agricultural facility floor and a module docking location adjacent a transfer station or dosing station. As appreciated by one of skill in the art having the benefit of this disclosure, a module can define any other suitable structure or geometry and can define any other suitable number or arrangement of plant slots.

### Water Sampling System

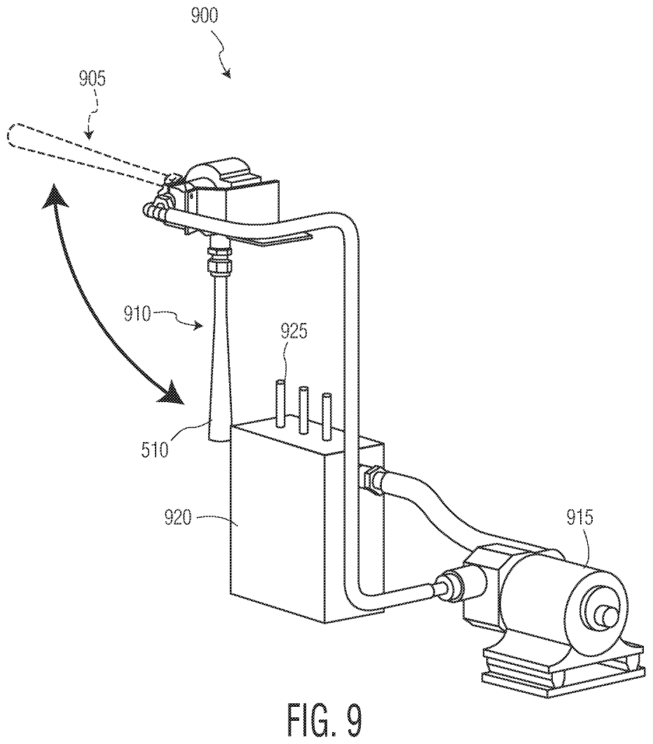

FIG. 9 illustrates a simplified view of one example of a water sampling system 900 that can be used in dosing station 200, according to embodiments of the disclosure. As shown in FIG. 9, water sampling system 900 includes sample pipe 510 that can rotate from a retracted position 905 to an extended position 910 that can access water within a module 205 (see FIG. 6). A pump 915, such as for example a peristaltic pump, can be operatively coupled to sample pipe 510 such that when operated it can draw water from a module into sample reservoir 920. A plurality of sensors 925 can be immersed in the water within sample reservoir to detect nutrient level, electrical resistivity, pH, oxygenation, bacteria count, temperature, a turbidity, an oxygen-reduction potential, a chlorine residual, and/or a total organic carbon value or other suitable parameters. In some embodiments sample reservoir 920 may maintain enough water to keep plurality of sensors 925 immersed, even when dosing station 200 is off-line. Once the water has been sampled and tested, it can be sent to a recycling drain, back to the module or to a drain. Any suitable number of sequential samples can be taken, tested and analyzed to accurately determine one or more parameters of the water.

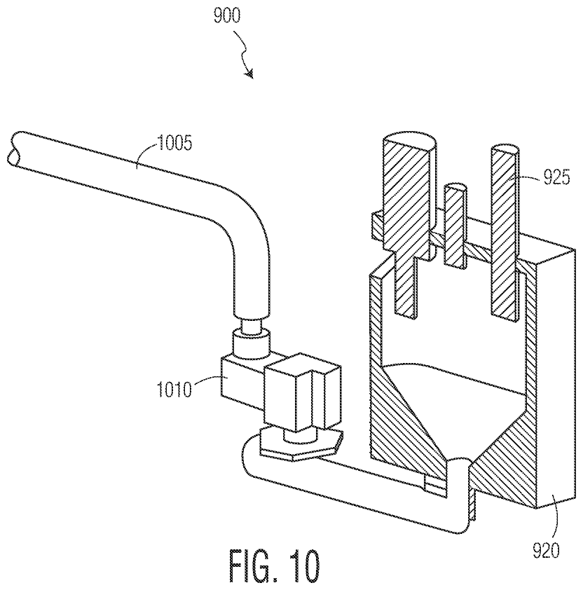

FIG. 10 illustrates a simplified cross-sectional view of a portion of water sampling system 900 including the sample reservoir, sensors and drain system. As shown in FIG. 10, sample reservoir 920 can include a plurality of sensors 925 that can test a water sample acquired from a module 205. Each sensor 925 can generate one or more outputs related to one or more parameters of the water that can be used by dosing station 200 or an ancillary computing system to determine an optimum nutrient recipe for the module. After the water sample has been analyzed the reservoir can be drained via drain pipe 1005 and valve 1010 and optionally flushed and/or refilled.

In some embodiments plurality of sensors 925 can include any number of or combination of: a pH sensor that outputs a signal representing pH of a water sample contained in the sample reservoir; an electrical conductivity sensor (e.g., including a pair electrodes) that outputs a signal representing nutrient level present in the water sample; a temperature sensor that outputs a signal corresponding to a temperature of the water; a turbidity sensor that outputs a signal representing suspended solids in the water sample; an oxygen-reduction potential (or “ORP”) sensor that outputs a signal representing a level of oxidation/reduction reactions occurring in the water sample; a chlorine residual sensor that outputs a signal representing total chlorine in the water sample; and/or a total organic carbon (or “TOC”) sensor that outputs a signal representing a carbon content of the water sample.

In some embodiments dosing station 200 and/or the ancillary computing system can: acquire signals from the set of sensors 925 in the sample reservoir 920; and evaluate water quality of the sample, related to the water quality in the module, based on these sensor data. For example, the dosing station 200 can capture: a relative nutrient level (or conductivity), a pH, a temperature, a turbidity, an oxygen-reduction potential, a chlorine residual, and/or a total organic carbon value of the water sample; and a water level in the module.

In some embodiments, sensors 925 can be arranged in a base of the sample reservoir 920 such that these sensors remain bathed in water even if the water level in the sample reservoir is low, thereby reducing opportunity for these sensors (e.g., especially the pH sensor) to dry out between refill cycles at the dosing station, which may affect calibration of these sensors. In various embodiments dosing station 200 can include multiple, redundant instances of sensors 925 and can discard anomalous results from redundant groups of like sensors and/or can average results from groups of like sensors when characterizing water quality in a module during a refill cycle, such as described below.

In some embodiments dosing station 200 stores a water sample—drawn from another module during the preceding refill cycle in the sample reservoir 920 between refill cycles. This previous water sample can function as a water bath for sensors 925 in the dosing station and may prevent the sensors from drying out between refill cycles. Furthermore, because the sample reservoir 920 remains full of this previous water sample, interior walls of the sample reservoir may also remain immersed in water rather than be exposed to air between refill cycles, which may further inhibit microbial growth within the sample reservoir between refill cycles.

In various embodiments, before drawing a new water sample from a new module in the receiving area, the dosing station can: empty the previous water sample into the waste water tank; and flush the sample reservoir (e.g., with water from the module rather than with fresh water in order to prevent dilution of the new water sample). The dosing station can then draw the new water sample out of the new module and into the sample reservoir.

In some embodiments, during an initial drain sub-cycle, the dosing station can: actuate a sump valve to open the inlet of the sample pipe to ambient air; actuate a reservoir valve to close the sample reservoir to ambient; actuate a waste water tank valve to open the sample reservoir to the waste water tank; and actuate the sample pump to pump air into the sample reservoir and thus displace the previous water sample into the waste water tank via a drain in the bottom of the sample reservoir. In various embodiments, the dosing station can: actuate the sample pump for a fixed duration of time corresponding to complete removal of water from the sample reservoir; track a fill level in the sample reservoir, such as via a reservoir fill level sensor (e.g., a depth sensor, a float sensor), and actuate the sample pump until the fill level drops below a minimum fill level; or track a fluid flow rate through the drain, such as via a flow rate sensor arranged between the drain and the waste water tank, and actuate the sample pump until this flow rate drops below a threshold minimum flow rate.

During a first fill sub-cycle, the dosing station can: trigger the sump actuator to lower the sample pipe into the module; actuate the reservoir valve to open the sample reservoir to ambient; actuate the waste water tank valve to close the sample reservoir to the waste water tank; and actuate the sample pump to pump water from the module into the sample reservoir and thus displace air out of the sample reservoir. In this embodiment, the dosing station can: actuate the sample pump for a fixed duration of time corresponding to a minimum fill level in the sample reservoir; or track a fill level in the sample reservoir, such as via the reservoir fill level sensor, and actuate the sample pump until the fill level reaches a maximum fill level.

The dosing station can then: repeat the foregoing process during a second drain sub-cycle to displace this new volume of water out of the sample reservoir and into the waste water tank, thereby flushing the sample reservoir with a volume of water exhibiting the same pH and dissolved nutrients as the water sample subsequently drawn from the module; and repeat the foregoing process during a second fill sub-cycle to refill the sample reservoir with a new water sample from the module. In this example, the dosing station can execute discrete drain, fill, empty, and fill sub-cycles to flush the sample reservoir and then capture a new water sample from the module.

In some embodiments, upon detecting that the sample reservoir is full during the fill sub-cycle, the dosing station can: actuate the reservoir valve to close the sample reservoir to ambient; actuate the waste water tank valve to open the sample reservoir to the waste water tank; and continue to actuate the sample pump to pump water from the module into the sample reservoir, an equal volume of which then flows down the reservoir drain and thus into the waste water tank. In this embodiment the dosing station can execute a drain sub-cycle and then execute concurrent flush and fill sub-cycles to flush the sample reservoir while capturing a new water sample from the module, which may reduce the total water removed from the module a) to flush the sample reservoir and b) to test water quality in the module during this refill cycle.

### Sample Pipe Sanitization

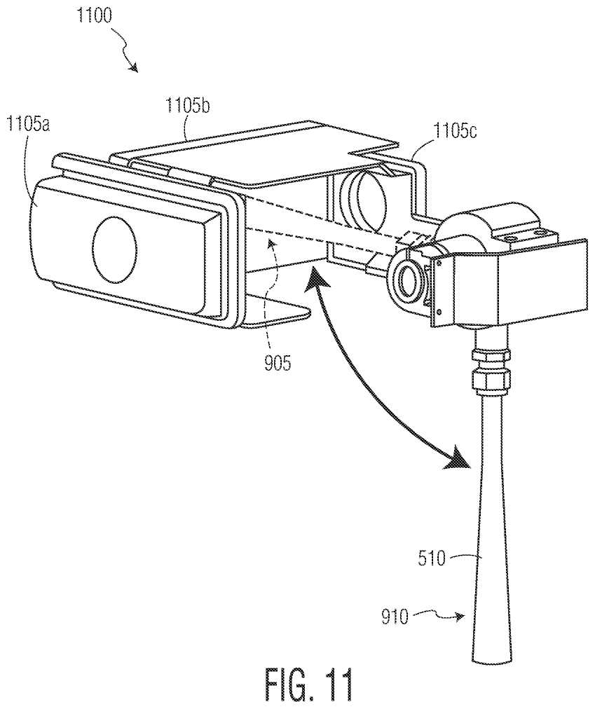

FIG. 11 illustrates a simplified view of a sample pipe cleaning system 1100 that can be used in dosing station 200, according to embodiments of the disclosure. As shown in FIG. 11, cleaning system 1100 can include three UV sanitization modules 1105a, 1105b, 1105c that clean sample pipe when it is in retracted position 905. More specifically, to clean substantially all surfaces of sample pipe 510 that were in contact with the water within the module, UV sanitization modules 1105a and 1105c can clean the exterior surfaces while UV sanitization module 1105b is axially aligned with sample pipe 510 to clean the interior of the sample pipe. To facilitate cleaning the interior of sample pipe 510, the sample pipe may have a tapered geometry so UV sanitization module 1005b can access the interior surface. In some embodiments an internal angle of sample pipe 510 can be between 70-90 degrees while in other embodiments it can be between 80-89 degrees and in one embodiment can be approximately 88 degrees.

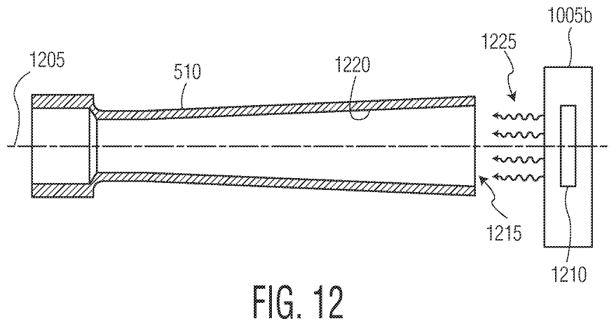

FIG. 12 illustrates a simplified cross-sectional view of sample pipe 510 in a retracted position during a cleaning operation. As shown in FIG. 12, UV sanitization module 1105b is axially aligned with sample pipe along an axis 1205. A UV source 1210 emits UV light 1225 that enters open end 1215 of sample pipe 510 and sanitizes interior surface 1220. In some embodiments UV source 1210 can be activated once, intermittently, or continuously in order to inhibit microbial growth on the sample pipe between refill cycles.

In some embodiments dosing station 200 can also include a sanitization subsystem (not shown) configured to receive and sanitize the sample pipe between refill cycles. In particular, the sanitization subsystem can sanitize the sample pipe between refill cycles in order to prevent transfer of pests (e.g., bacteria, mold, viruses, insects) between modules due to contact between the sample pipe and water contained in these modules during refill cycles. The sanitization subsystem can include a sanitization vessel loaded with a solution bath (e.g., alcohol, steam). Between refill cycles, the system can position the sample pipe into the sanitization vessel, which bathes and sanitizes the sample pipe in the solution bath.

In some embodiments pump 915 (see FIG. 9) can be configured to draw water from the module 205 into the sample reservoir 920 via the sample pipe 510 and a filter can be arranged between the sample pipe and the sample reservoir. In one example, the sample pipe 510 can include a rigid tube arranged over and facing downwardly toward the module 205; and a flexible line extending from a top of the rigid tube to the pump 915. In this example, a sump actuator can include a set of (e.g., three) offset rollers that locate the rigid tube above the module 205 and that are operable to raise and lower the sample pipe 510 out of and into the module. Alternatively, in this example, the sump actuator can include a pneumatic rotary actuator that extends and retracts the rigid tube into and out of the module 205.

In various embodiments dosing station 200 can also include a set of valves that selectively couple and decouple the sample pump, the sample reservoir, a waste water tank, and an exhaust port to ambient to enable the singular sample pump to fill, empty, and flush the sample reservoir. In some embodiments dosing station 200 can include multiple pumps, such as including: a first pump configured to pump water from the sample pipe to the sample reservoir; and a second pump configured to pump water from the sample reservoir to the waste water tank.

### Refill Recipe

In some embodiments dosing station 200 (see FIG. 2A) can retrieve a target water quality assigned to the set of plants occupying the module 205, such as from a grow schedule stored in a local or remote database. Alternatively, dosing station 200 can implement methods and techniques described herein to calculate a custom target water quality (e.g., target conductivity, pH, turbidity, oxygen-reduction potential, chlorine residual, and/or total organic carbon values) and a target total water level for the module, such as, based on a species of the plants in the module, based on an age of plants in the module, past environmental exposure of these plants, and/or measured qualities of the water in the module.

In various embodiments, dosing station 200 can: calculate a total actual water volume in the module 205 based on the water fill level (e.g., by inserting the water fill level of the module into a volume function that returns a total actual water volume in the module); calculate a total actual amount of nutrients in the module based on the relative nutrient level (or the conductivity) of the water sample and the total actual water volume in the module; calculate a total target amount of nutrients in the module based on the target nutrient level and the total target water volume in the module; calculate a difference between the total actual amount and the total target amount of nutrients in the water; and store this difference as a nutrient dispense volume.

In some embodiments dosing station 200 can: calculate a difference between the total actual water volume and the total target water volume in the module and store this difference as a water dispense volume; estimate an intermediate pH of water in the module following addition of the nutrient dispense volume and the water dispense volume to the module based on the current actual pH in the module; and calculate a difference between the intermediate pH and the target pH for the module. Then, if the intermediate pH is greater than the target pH, the dosing station can calculate a dispense volume of pH reducer that will reduce the actual pH in the module to the target pH based on the intermediate pH and the total target water volume in the module. Conversely, if the intermediate pH is less than the target pH, the dosing station can calculate a dispense volume of alkaline solution that will increase the actual pH in the module to the target pH based on the intermediate pH and the total target water volume in the module.

In various embodiments dosing station 200 can calculate: an amount of water in the module to remove and replace, such as based on target and actual turbidity, oxygen-reduction potential, chlorine residual, and/or total organic carbon values of water in the module; and calculate a target change in water level in the module to remove this amount of water from the module. The dosing station can then: actuate the reservoir valve to close the sample reservoir to ambient; actuate the waste water tank valve to open the sample reservoir to the waste water tank; actuate the sample pump to pump water from the module into the sample reservoir; monitor the water level in the module; and then deactivate the sample pump once the water level in the module changes by the target change in water level. The dosing station can then implement methods and techniques described above to recalculate amounts of water, nutrients, and pH modifier to add to the module.

In some embodiments, dosing station 200 retrieves a fixed nutrient recipe specifying a target pH and a target nutrient level (or electrical conductivity, etc.) for the module based on a stage of plants in the module (e.g., seeding, nursery, or finishing stages), an average age (e.g., in days) of plants in the module, and/or or a predefined grow and nutrient scheduled assigned to these plants. For example, a remote computer system can implement methods and techniques described in U.S. patent application Ser. No. 17/028,641 to derive a target pH and a target nutrient level (or conductivity, etc.) for plants of the same type and at the same stage or age as plants in the module that yield harvestable plants of target characteristics (e.g., size, color, flavor). The dosing station can then implement methods and techniques described above to realize these target pH and nutrient levels in the module.

In various embodiments, the robotic raft manipulator or a separate imaging station includes an optical sensor captures images of plants in the module. In some embodiments a fixed optical sensor located overhead the module dock at the dosing station can capture images of plants in the module once delivered to the dosing station. In various embodiments the mover can navigate the module through an imaging station in the agricultural facility before delivering the module to the dosing station in preparation for a scheduled refill cycle, and the imaging station can capture images of plants in the module. In this implementation, the auxiliarry computer system (or the dosing station) can then implement methods and techniques described in U.S. patent application Ser. Nos. 17/129,130 and 17/028,641 to: access these images of plants in the module; detect plants in these images; extract features representative of these plants from these images; and derive characteristics of these plants from these features, such as size, shape, foliage fill factor, color, pest presence or pest indicators, heat burn, chemical burn, nutrient deficiency, water deficiency, and/or root rot. The auxiliary computer system (or the dosing station) can then calculate a module-specific nutrient recipe based on these plant characteristics and a plant nutrient module.

For example, the computer system can: set a lower target water level in response to detecting root rot in these plants; set a higher target water level in response to detecting water deficiency in these plants; set a higher target nutrient level if no chemical burns are detected in these plants but these are plants undersized or exhibiting low or non-uniform foliage fill factors; and/or set a lower target nutrient level if chemical burns are detected in these plants; etc. In another example, in this implementation, the remote computer system (or the dosing station) can implement a nutrient module, derived for example according to methods and techniques described in U.S. patent application Ser. No. 17/028,641, to convert visual characteristics of plants in the module into target nutrient, pH, and water levels in the module. The dosing station can then implement methods and techniques described above to realize these target pH and nutrient levels in the module.

### Nutrient Dispensing System

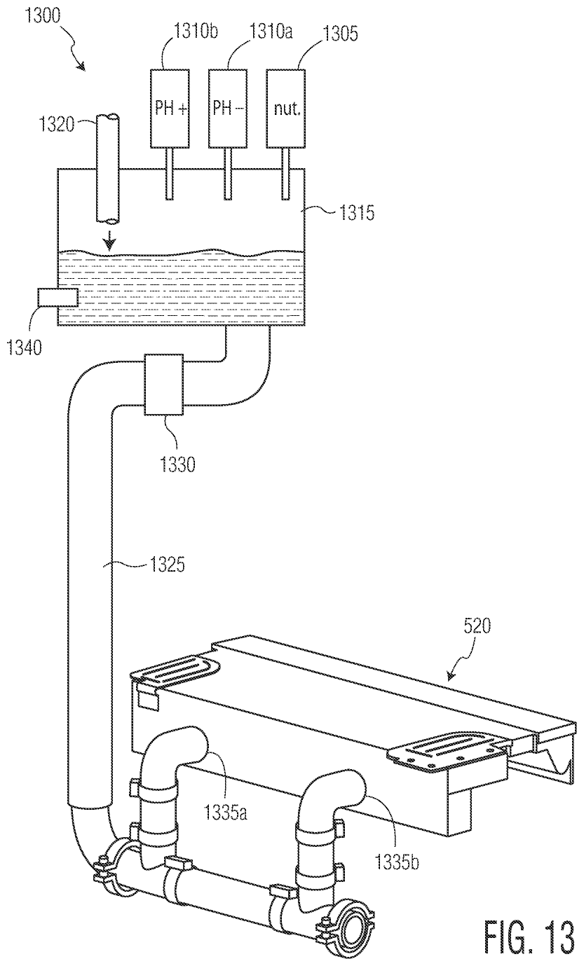

FIG. 13 illustrates a simplified view of an example nutrient dispensing system that can be used in dosing station 200, according to embodiments of the disclosure. As shown in FIG. 13, nutrient dispensing system 1300 includes one or more nutrient dispensers 1305, one or more pH adjustment dispensers 1310 and can include other suitable types of dispensers. In one embodiment nutrient dispensing system includes two separate nutrient dispensers and two pH dispensers 1310a, 1310b (e.g., one for increasing pH and one for decreasing pH). In some embodiments nutrient dispensing system 1300 may include a premix tank 1315 in which one or more of the dispensed compounds may be mixed before being discharged into the module 205 (see FIG. 2A) however in other embodiments the one or more dispensed compounds may be dispensed in a discharge tube with water that is used to refill the module. Premix tank 1315 may include a water inlet 1320 and may include a sample port 1340 for sampling the premix solution to determine if it has the appropriate levels of nutrients and pH. In further embodiments premix tank 1315 itself may include one or more sensors (e.g., installed in a sidewall of the premix tank) that sense parameters of the premix solution while in the premix tank so adjustments to the premix solution can be made before discharging the solution to a module. Nutrient dispenser 1305 and pH dispensers 1310a, 1310b may dispense liquids, gels, powders or any other suitable form of compounds.

Nutrient dispenser 1305 and pH dispensers 1310a, 1310b may be automatically controlled via a controller within dosing station 200 or may be controlled via an ancillary computing system. In some embodiments, the outputs from plurality of sensors 925 (see FIG. 9) in sample reservoir 920 may be used to determine an appropriate amount of nutrient to be dispensed by nutrient dispenser 1305 and additives to be dispensed by pH dispensers 1310a, 1310b, which may each may have suitable automatic controls that can meter the respective compounds into mixing tank 1315.

In some embodiments mixing tank 1315, nutrient dispenser 1305 and pH dispensers 1310a, 1310b may form a part of a physically separate apparatus from the portion of the dosing station that receives and interacts with modules 205, however, the two separate apparatus may together form the dosing station 200 described herein. In some embodiments nutrient dispensing system 1300 is coupled to spout 520 via dispensing pipe 1325 which is controlled by drain valve 1330. In various embodiments dispensing pipe 1325 may engage with one or more entry ports 1335a, 1335b to maximize laminar flow into spout 520.

In some embodiments dosing station 200 includes: a set of nutrient storage containers; a fresh water valve coupled to a freshwater supply; a nutrient premixing chamber; a set of nutrient pumps configured to meter nutrients from the nutrient containers into the nutrient premixing chamber; a mixing head configured to agitate water and nutrients loaded into the nutrient premixing chamber; and a chamber pump configured to pump a nutrient solution out of the nutrient premixing chamber and into a module, such as via the spout described above.

For example, in some embodiments the premixing tank 1315 can be teed into a freshwater line between the freshwater valve and the spout, and the chamber pump can pump a premixed nutrient solution from the premixing chamber into the freshwater line for further mixing with freshwater upstream of the spout and before release into the module. In various embodiments, the nutrient pumps can: meter doses of nutrients from their storage containers directly into the freshwater line between the freshwater value and the spout, and these nutrients can then mix with freshwater upstream of the spout and before reaching the module.

In some embodiments the dosing station 200: monitors the fill level in the module via the fill level sensor; and triggers a freshwater valve to open to release fresh water into the module. During this period, the dosing station can also actuate the nutrient pump (or valves connected to the nutrient containers) to meter the nutrient dispense volume of a nutrient solution, a dispense volume of pH reducer, and/or a dispense volume of alkaline solution into the module. For example, the dosing station can release these nutrients into the freshwater line upstream of the spout such that these nutrients are mixed into this stream of water and are diluted before entering the module. The dosing station can then trigger the freshwater valve to close once the fill level sensor indicates the target fill level in the module.

In various embodiments, the dosing station 200 actuates nutrient pumps (or valves) to dispense the nutrient dispense volume of a nutrient solution, a dispense volume of pH reducer, and/or a dispense volume alkaline solution into the nutrient premixing chamber described above. For example, the dosing station can include a scale coupled to the nutrient premixing chamber. The dosing station can: tare the scale before dispensing a first nutrient into the nutrient premixing chamber; actuate a first nutrient pump to dispense a first nutrient into the nutrient premixing chamber until the scale reads a first target weight for this first nutrient; and then repeat this process for each other nutrient designated for addition to the module during this refill cycle.

In some embodiments, when dispensing a nutrient into the nutrient premixing chamber, the dosing station can track an amount of the nutrient based on an output of a mass or volume flow rate sensor coupled to the nutrient container. In various embodiments, when dispensing a nutrient into the nutrient premixing chamber, the dosing station can: calculate an actuation duration for the nutrient based on known flow rate of a nutrient pump connected to the nutrient container and the target amount of the nutrient allocated for the module; and then actuate the nutrient pump for this actuation duration. In some embodiments the dosing station 200 can actuate the freshwater valve to release freshwater from the freshwater supply into the nutrient premixing chamber, thereby diluting these nutrients (e.g., to prevent root burn when these nutrients are initially dispensed into the module); and then actuate the chamber pump to dispense this nutrient solution into the module.

In various embodiments the dosing station 200 can repeat the process described above to: capture a second water sample from the module after completing the preceding refill cycle; test water quality of this second water sample; and verify that the water quality of the second water sample falls within a threshold tolerance of the target water quality. The dosing station can then repeat the preceding process to add additional nutrients or freshwater to the module if the water quality of the second water sample falls outside of the threshold tolerance of the target water quality.

### Spout Assembly

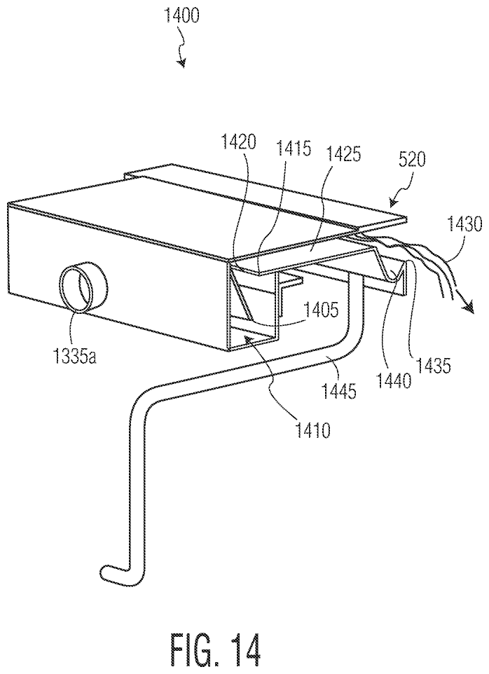

FIG. 14 illustrates a simplified cross-sectional view of an example spout assembly 1400 that can be used with dosing station 200. As shown in FIG. 14, spout assembly 1400 includes one or more ports 1335a that receive nutriated water from mixing tank 1315 (see FIG. 13). To minimize filling time of modules 205 (see FIG. 2A) while maintaining controlled laminar flow into the modules to minimize splashing, spout assembly 1400 may have multiple ports 1335a to receive large quantities of nutriated water. Spout assembly 1400 may also have an inlet deflector 1405 arranged to deflect incoming nutriated water into receiving trough 1410. A trough baffle 1415 may form a reduced trough exit 1420 into a laminar flow channel 1425 that terminates at spout 520 where nutriated water exits in a broad laminar stream 1430. Spout assembly 1400 may also include a drip ledge 1435 that forms a drip tray 1440 that is connected to a drain via drain tube 1445. In some embodiments spout assembly can deliver 40-70 gallons per minute with a laminar flow.

In some embodiments spout 520 can define a linear filler extending laterally across the module 205 by a length approximating (e.g., 90% of) a standard raft 315 width. Dosing station 200 can manipulate spout 520 over and parallel to the adjacent edge of the module 205, and the spout can dispense water into the module along this adjacent edge of the module, which may limit splashing within the module (e.g., relative to a circular stream of water dispensed into the module).

### Module Draining

In some embodiments dosing station 200 can use similar methods and techniques as described above to empty a module, such as, before or after a set of plants are removed (e.g., harvested) from the module. In various embodiments, a module can include a sump trough extending along its base and defining a low point in the module at a position aligned with the sample pipe when the module is located in the module dock at the dosing station. Thus, upon receiving a module designated for emptying, cleaning, and sanitation, the dosing station can: trigger the sump actuator to lower the sample pipe down into the trough in the tray of the module; actuate the reservoir valve to close the sample reservoir to ambient; actuate the waste water tank valve to open the sample reservoir to the waste water tank; actuate the sample pump to pump water from the module into the sample reservoir; monitor the water level in the module; and then deactivate the sample pump once the water level in the module reaches a minimum water level or until a volume flow rate into the sample reservoir drops below a threshold volume flow rate. The mover can then autonomously deliver the module to a clearing station, such as described in U.S. patent application Ser. No. 16/898,784. In this example, the dosing station can also: include an optical sensor; detect the trough in the tray via the optical sensor; and trigger the sump actuator to move the sample pipe laterally and/or longitudinally to align with and enter the trough, thereby enabling the sample pipe to fully drain the tray.

### Sump/Spout Connection

In some embodiments a module 205 includes: a spout connector; a spout trap; a sump connector; and a sump trap. The spout connector is arranged on a bottom or side of the tray and is configured to couple to and seal against the spout of the dosing station. The spout trap: extends from the spout connector when the spout connector is disconnected from the spout; extends above a maximum water line in the tray to prevent water from flowing from the tray back through the spout connector; and loops downwardly to dispense water—flowing from the dosing station into the spout connector—into the tray.

The sump connector can be arranged on a bottom or side of the tray and is configured to couple to and seal against the sample pipe of the dosing station. The sump trap: extends from the sump connector; extends above a maximum water line in the tray to prevent water from flowing from the tray back through the sump connector when the sump connector is disconnected from the sample pipe; loops downwardly toward the tray; and terminates just about a trough or other low region in the base of the tray. In this example, the dosing station can selectively couple to the spout and sump connectors to dispense water into the module and extract a water sample from the module, respectively, without necessitating removal of a raft from the module to access the volume of water contained therein.

In some embodiments dosing station 200 can also include a waste water tank (not shown) configured to store water samples drawn out of modules 205 during refill cycles. For example, the waste water tank can include a 150-gallon tank or other suitably sized tank, which may be contain waste water drawn during: refill cycles at 25 modules; and module empty cycles at two 50-gallon modules.

Dosing station 200 can also include a water supply valve (not shown) connected to a fresh water supply, such as to a fresh water holding tank arranged in the agricultural facility and preloaded with water: previously recycled from modules in the agricultural facility by the dosing station and/or filtered, and then sanitized, as described below.

In some embodiments, dosing station 200 also includes a waste water pump (or interfaces with another external pump within the agricultural facility) configured to move waste water from the waste water tank to a waste water recycler within the agricultural facility. The waste water recycler can then: filter and sanitize this waste water (e.g., via electrochemically-activated water, or “ECA”); and return a stream of fresh (e.g., potable) recycled water directly to the dosing station or store this fresh water in a fresh water holding tank connected to the dosing station. The waste water recycler can also extract nutrients from the waste water and package these nutrients for recycling or distribution back to the dosing station.

The systems and methods described herein can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives or any suitable device. The computer-executable component can be a processor but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

In the foregoing specification, embodiments of the disclosure have been described with reference to numerous specific details that can vary from implementation to implementation. The specification and drawings are, accordingly, to be regarded in an illustrative rather than a restrictive sense. The sole and exclusive indicator of the scope of the disclosure, and what is intended by the applicants to be the scope of the disclosure, is the literal and equivalent scope of the set of claims that issue from this application, in the specific form in which such claims issue, including any subsequent correction. The specific details of particular embodiments can be combined in any suitable manner without departing from the spirit and scope of embodiments of the disclosure.

Additionally, spatially relative terms, such as “bottom or “top” and the like can be used to describe an element and/or feature's relationship to another element(s) and/or feature(s) as, for example, illustrated in the figures. It will be understood that the spatially relative terms are intended to encompass different orientations of the device in use and/or operation in addition to the orientation depicted in the figures. For example, if the device in the figures is turned over, elements described as a “bottom” surface can then be oriented “above” other elements or features. The device can be otherwise oriented (e.g., rotated 90 degrees or at other orientations) and the spatially relative descriptors used herein interpreted accordingly.

Terms “and,” “or,” and “an/or,” as used herein, may include a variety of meanings that also is expected to depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein may be used to describe any feature, structure, or characteristic in the singular or may be used to describe some combination of features, structures, or characteristics. However, it should be noted that this is merely an illustrative example and claimed subject matter is not limited to this example. Furthermore, the term “at least one of” if used to associate a list, such as A, B, or C, can be interpreted to mean any combination of A, B, and/or C, such as A, B, C, AB, AC, BC, AA, AAB, ABC, AABBCCC, etc.

Reference throughout this specification to “one example,” “an example,” “certain examples,” or “exemplary implementation” means that a particular feature, structure, or characteristic described in connection with the feature and/or example may be included in at least one feature and/or example of claimed subject matter. Thus, the appearances of the phrase “in one example,” “an example,” “in certain examples,” “in certain implementations,” or other like phrases in various places throughout this specification are not necessarily all referring to the same feature, example, and/or limitation. Furthermore, the particular features, structures, or characteristics may be combined in one or more examples and/or features.

In some implementations, operations or processing may involve physical manipulation of physical quantities. Typically, although not necessarily, such quantities may take the form of electrical or magnetic signals capable of being stored, transferred, combined, compared, or otherwise manipulated. It has proven convenient at times, principally for reasons of common usage, to refer to such signals as bits, data, values, elements, symbols, characters, terms, numbers, numerals, or the like. It should be understood, however, that all of these or similar terms are to be associated with appropriate physical quantities and are merely convenient labels. Unless specifically stated otherwise, as apparent from the discussion herein, it is appreciated that throughout this specification discussions utilizing terms such as “processing,” “computing,” “calculating,” “determining,” or the like refer to actions or processes of a specific apparatus, such as a special purpose computer, special purpose computing apparatus or a similar special purpose electronic computing device. In the context of this specification, therefore, a special purpose computer or a similar special purpose electronic computing device is capable of manipulating or transforming signals, typically represented as physical electronic or magnetic quantities within memories, registers, or other information storage devices, transmission devices, or display devices of the special purpose computer or similar special purpose electronic computing device.

In the preceding detailed description, numerous specific details have been set forth to provide a thorough understanding of claimed subject matter. However, it will be understood by those skilled in the art that claimed subject matter may be practiced without these specific details. In other instances, methods and apparatuses that would be known by one of ordinary skill have not been described in detail so as not to obscure claimed subject matter. Therefore, it is intended that claimed subject matter not be limited to the particular examples disclosed, but that such claimed subject matter may also include all aspects falling within the scope of appended claims, and equivalents thereof.

What is claimed is:

1. A dosing station comprising:
a receiving area for receiving a module;
a sampling system for acquiring water from the module, analyzing the water and determining a refill recipe based on one or more parameters of the water; and
a nutrient dispenser arranged to dispense at least one nutrient into the module according to the refill recipe.

2. The dosing station of claim 1 wherein the sampling system includes an electrical resistivity probe.

3. The dosing station of claim 1 wherein the sampling system includes an intake pipe that is arranged to be immersed in the water in the module.

4. The dosing station of claim 3 further comprising a sanitization system arranged to sanitize the intake pipe after the immersing.

5. The dosing station of claim 4 wherein the sanitization system comprises an ultraviolet source.

6. The dosing station of claim 5 wherein the intake pipe is tapered and the ultraviolet source is axially aligned with the intake pipe.

7. The dosing station of claim 1 wherein the module is a first module and the receiving area is arranged to receive a second module.

8. The dosing station of claim 1 wherein the refill recipe is determined at least in part from an optical image of one or more plants in the module.

9. A method of refilling a grow module in an agricultural facility, the method comprising:
positioning the grow module adjacent a dosing station;
acquiring a water sample from the grow module with a sampling system, testing the water sample and generating an output based on the water sample;
determining a grow module refill recipe based on the output; and
dispensing a nutrient into the grow module based on the refill recipe.

10. The method of claim 9 wherein the sampling system includes an electrical resistivity probe.

11. The method of claim 9 wherein the sampling system further comprises an intake pipe that extends into the grow module to acquire the water sample.

12. The method of claim 11 further comprising sanitizing the intake pipe after acquiring the water sample.

13. The method of claim 9 wherein the refill recipe is determined at least in part based on a type of plant in the grow module.

14. The method of claim 9 wherein the refill recipe is determined at least in part from an optical image of one or more plants in the grow module.

15. A method of growing plants in a grow module within an agricultural facility, the method comprising:
positioning the grow module in a growth area of the agricultural facility;
transporting the grow module to a dosing station;
acquiring a water sample from the grow module with a sampling system, testing the water sample and generating an output based on the water sample;
determining a module refill recipe based on the output;
dispensing a nutrient into the grow module based on the refill recipe; and
transporting the grow module to the growth area.

16. The method of claim 15 wherein the transporting is performed by an autonomous mover.

17. The method of claim 15 wherein the refill recipe is additionally based on a species of the plants in the grow module.

18. The method of claim 15 wherein the refill recipe is additionally based on an image of the plants in the grow module.

19. The method of claim 15 wherein the output indicates a nutrient content in the water sample.

20. The method of claim 15 wherein the output indicates a pH of the water sample.
